(19) United States
(12) Patent Application Publication
(10) Pub. No.: US 2010/0268039 A1
(43) Pub. Date: Oct. 21, 2010

(54) SYSTEM FOR DIAGNOSING REAL-TIME PHYSIOLOGICAL SIGNAL

(75) Inventors: EN JEN, CHUNG LI (TW); YI-JHEN WU, CHUNG LI (TW); GUAN-YING CHEN, CHUNG LI (TW)

Correspondence Address: ROSENBERG, KLEIN & LEE, 3458 ELLICOTT CENTER DRIVE-SUITE 101, ELLICOTT CITY, MD, 21043, US

(73) Assignee: CHUNG YUAN CHRISTIAN UNIVERSITY, CHUNG LI (TW)

(21) Appl. No.: 12/477,461

(22) Filed: Jun. 3, 2009

(30) Foreign Application Priority Data
Apr. 16, 2009 (TW) 098112646

(51) Int. Cl. A61B 5/00

(52) U.S. Cl. 600/301

(57) ABSTRACT

A diagnose system includes a signal-capturing device and a real-time diagnose device coupled electrically to the signal-capturing device. The signal-capturing device includes a signal-receiving interface for acquiring a physiological signal of a person, a micro processing unit for processing the physiological signal and promptly converting into a physiological parameter, and a wireless transceiver module for transmitting the physiological parameter. The real-time diagnose device includes a wireless reader for receiving the physiological parameter and a memory unit including a distinguishing database and a prescription database. The distinguishing database is stored with a plurality of standard physiological parameters for judging. The prescription database is stored with several prescribed treatments, one of which is corresponding to the physiological parameter when the physiological parameter is abnormal, and a processor coupled electrically to the reader and the memory unit for promptly analyzing the distinguishing database, thereby distinguish whether the physiological parameter is normal or not.

This application claims the benefit of the Taiwan Patent Application Serial NO. 098112646, filed on Feb. Apr. 16, 2009, the subject matter of which is incorporated herein by reference.

### BACKGROUND OF THE INVENTION

1. Field of the Invention

The present invention relates to a real-time physiological signal diagnose system, more particularly to a diagnose system in which, the physiological signal of a patient is promptly diagnosed and the diagnosis resulting thereby is displayed promptly in a display unit of the diagnose system.

2. Description of the Prior Art

Health condition of our people living in Taiwan improves lately due to rising living standard so that population of aged is on the rise continuously. Thus, Taiwan faces an old-aged community composed of elders beyond 65 years. Presently, the old-aged community occupies 7% of the whole population. What we now face is an urgent problem of how to look after these elders in case they are not provided with appropriate care at homes by their families.

The older the people get, the easier for them to be stricken with chronic disease due to gradual decrease of resistance. The physiological signals of these elders should be observed frequently. In order to maintain the good health, there should be certain caretakers to take them regularly to medical centers for check up and bring back home after treatment. Since the majority of the elders are not properly looked after at homes by their families, they are entrusted to the sanatorium or medical centers for attending to their needs. Some family faces financial problem in case their elder is entrusted to the sanatorium or medical center.

Of late ten years, several remote-control health care systems or detection system have been invented, provide relatively nice medical cares so that such detection systems partially share the high medical expenses and reduce the burden of the medical staff, thereby saving undesired medical expenses. Long time and continuously monitoring the health condition of the elder can notice the abnormal symptom of the patient. Therefore, taking good care at home plays an important part to protect the elder from being stricken with disease

ROC (Republic of China) Patent Publication No. 200417355 discloses “a wireless medical device for collecting real-time physiological signals”, which is adapted to download the physiological signal in a short time to a monitor system for undergoing analysis so as to record and monitor the data, thereby activating the alarm device for reminding the medical staff in case the data is abnormal. Because the monitored physiological signals need to be temporarily stored for undergoing analysis and registering, in case of critical or emergency situation, it is hard to grab the prime time opportunity effectively for saving the patient's life.

In another ROC Patent Publication No. M328933, “a wireless transmission and diagnose device” is disclosed, in which, a conventional cabled measuring medical device is converted into a wireless ones, that is capable of capturing not only the physiological signal but also transmitting the same so as to be stored within a database for future application by the medical staff. However, during the period of transmitting the physiological signal to the database and in case the patient being monitored encounters an emergency case, he is left without an alarm device for reminding the ambient persons, thereby missing the golden opportunity for saving the patient's life.

Later, ROC Patent Publication No. 200808267 discloses “a medical care system” that provides proper care and reduces the waste of medical source. The system is capable of diagnosing in case of monitoring abnormal physiological signals of the patient kept under observation and promptly activate the alarm device to remind of the abnormal physiological signal. However, the aforesaid care system is applicable only on the one-to-one basis, the alarm device can only remind the abnormal physiological signal and is unable to advise the ambient persons how to treat the patient in time of critical and emergency in order to maintain the condition from getting worse, thereby saving the golden opportunity for saving the patient's life.

Though the prior art medical care systems and devices can provide some sort of medical care, but fail to provide prompt diagnose and the diagnosis is not promptly shown in the display unit of the system. In addition, the prior art is applicable only on one-to-one basis and unable to provide one system for multiple patients. Therefore, it is the object of the present invention to provide a diagnose system for continuously monitoring the physiological signals of the patient. In case the physiological signal turns abnormal, the alarm device will generate a reminding sound or symbol to the patient's family or the medical staff so as to swiftly attend to the needs of the patient. After undergoing analysis of the physiological signals, an appropriate treatment or prescription will be given to the patient in order to remedy the critical situation, thereby eliminating the drawbacks encountered during use of the prior art medical system or device. When the present diagnose system is applied, a relative amount of medical expense can be lowered.

### SUMMARY OF THE INVENTION

Since the prior art diagnose system or medical care system fails to provide prompt diagnose and the diagnosis is not immediately shown in a display unit, the object of the present invention is to provide a prompt diagnose system, in which, the captured physiological signal is promptly analyzed and the diagnosis or appropriate treatment resulting thereby is given to the patient (or the ambient persons) in order to meet the critical situation of the patient in time of emergency.

Therefore, a diagnose system of the present invention includes a signal-capturing device and a real-time diagnose device. The signal-capturing device includes a signal-receiving interface for acquiring a physiological signal of a person under observation, a micro processing unit coupled electrically to the signal-receiving interface for processing the physiological signal and promptly converting the physiological signal into a physiological parameter, and a wireless transceiver module coupled electrically to the micro processing unit for transmitting the physiological parameter. The real-time diagnose device is coupled electrically to the signal-capturing device and includes a wireless reader coupled electrically to the wireless transceiver module for receiving the physiological parameter and a memory unit including a distinguishing database and a prescription database. The distinguishing database is stored with a plurality of standard physiological parameters as reference for judging whether the physiological parameter is normal or abnormal. The prescription database is stored with a plurality of prescribed treatments, one of which is corresponding to the physiological parameter in case the latter is abnormal. A processor is coupled electrically to the wireless reader and the memory unit for promptly analyzing the distinguishing database, thereby distinguishing the physiological parameter received in the wireless reader. In case the physiological parameter is abnormal, the processor will select a respective prescribed treatment from the prescription database in response to the abnormal physiological parameter after comparing the abnormal physiological parameter with respect to the standard physiological parameters.

When the diagnose system of the present invention is compared to the prior art ones, the present diagnose system possesses the ability of promptly diagnosing and analyzing the physiological signal and promptly providing a treatment direction in time of emergency. In case the physiological signal is abnormal, a respective prescribed treatment will be given in addition to alarming the medical staff for attending to the needs of the patient.

### BRIEF DESCRIPTION OF THE DRAWINGS

Other features and advantages of the present invention will become more apparent in the following detailed description of the preferred embodiment of this invention, with reference to the accompanying drawings, in which:

FIG. 1 is a real-time physiological signal diagnose system of the present invention in application; and

FIG. 2 is a block diagram representing the real-time physiological signal diagnose system of the present invention.

### DETAILED DESCRIPTION OF THE PREFERRED EMBODIMENT

Since the real-time physiological signal diagnose system of the present invention is capable of directly capturing the physiological signal of the person under observation and is widely applied in emergency device. Thus, the following embodiment is described only for better understanding and the scope of the invention should not be limited thereto.

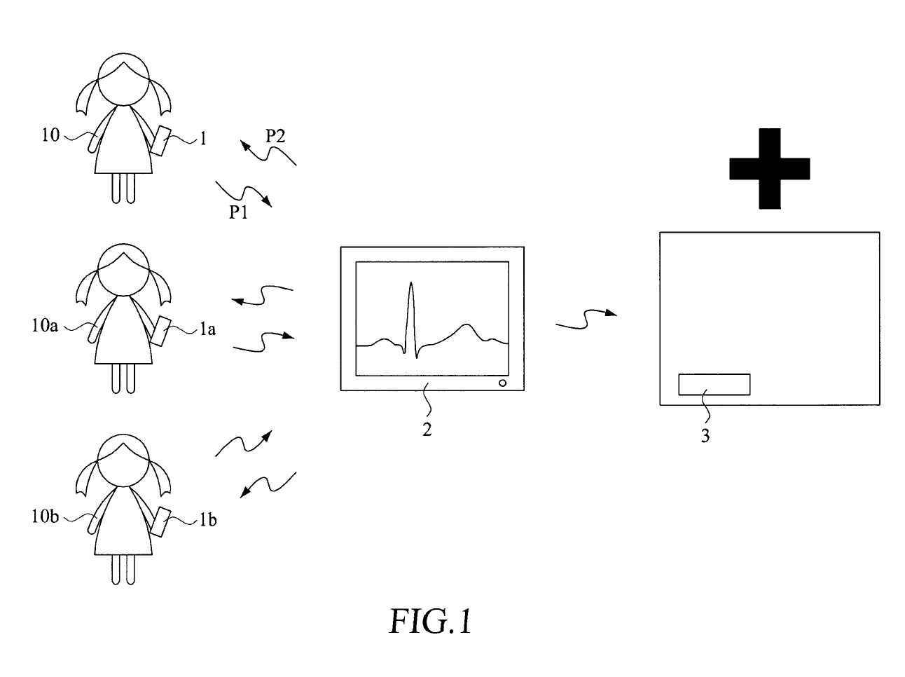

FIG. 1 shows a real-time physiological signal diagnose system 100 of the present invention in application, wherein the diagnose system 100 is applied to three persons (preferably patients) 10, 10a and 10b for monitoring, capturing and diagnosing the physiological signals thereof. The diagnose system 100 accordingly includes a real-time diagnose device 2 and three signal-capturing devices 1, 1a and 1b respectively fastened to the persons 10, 10a and 10b in such a manner to capture the physiological signals thereof.

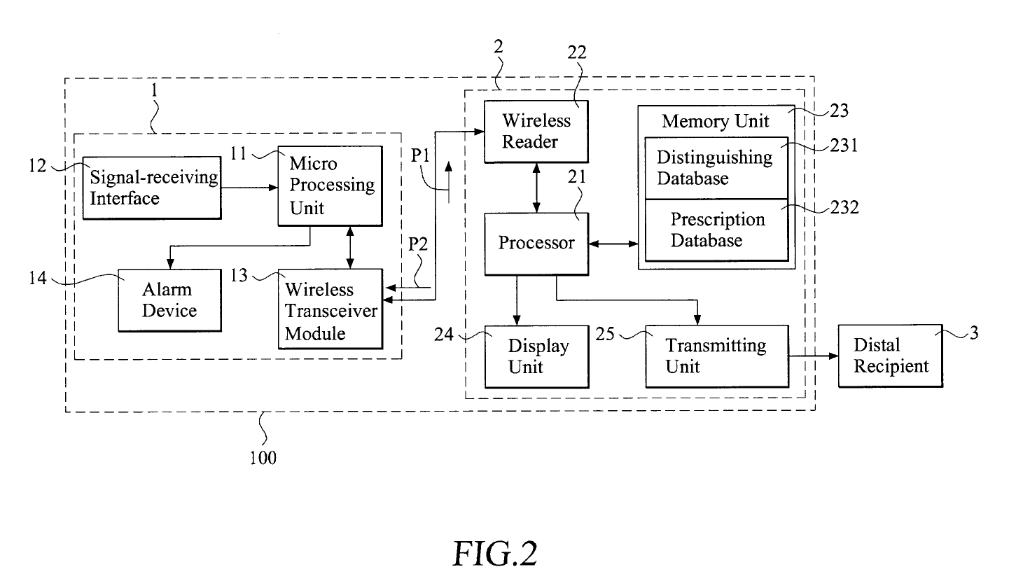

FIG. 2 is a block diagram representing the real-time physiological signal diagnose system of the present invention. As illustrated, each of the signal-capturing devices 1 includes a signal-receiving interface 12, a micro processing unit 11, a wireless transceiver module 13 and an alarm device 14. The signal-receiving interface 12, the wireless transceiver module 13 and an alarm device 14 are respectively and electrically connected to the micro processing unit 11.

The real-time diagnose device 2 includes a processor 21, a wireless reader 22, a memory unit 23, a display unit 24 and a transmitting unit 25. The wireless reader 22, the memory unit 23, a display unit 24 and a transmitting unit 25 are respectively and electrically connected to the processor 21. The memory unit 23 includes a distinguishing database 231 and a prescription database 232. The distinguishing database 231 is stored with a plurality of standard physiological parameters as reference for judging whether the physiological parameter P1 is normal or abnormal. The prescription database 232 is stored with a plurality of prescribed treatments, one of which is corresponding to the physiological parameter P1 in case the latter is abnormal.

Referring to FIGS. 1 and 2 again, the signal-receiving interface 12 of the signal-capturing device 1 is used for acquiring a physiological signal of the person 10 under observation and the physiological signal is later transmitted to the micro processing unit 11. The micro processing unit 11 is coupled electrically to the signal-receiving interface 12 for processing (such as magnifying, filtering, and converting from analog to digital) the physiological signal and promptly converting the physiological signal into the physiological parameter P1. The wireless transceiver module 13 is coupled electrically to the micro processing unit 11 for transmitting the physiological parameter P1 to the wireless reader 22 of the real-time diagnose device 22.

In the present invention, the physiological signal can be an ECG (Electrocardiogram) signal or a blood pressure of the person under observation or an amount of blood glucose of the person under observation. Preferably, the wireless transceiver module 12 and the wireless reader 22 can be a Radio Frequency Modem.

The wireless reader 22 is coupled electrically to the wireless transceiver module 12 for receiving the physiological parameter P1 and transfers the same to the processor 21 so that the physiological parameter P1 is displayed in the display unit 24. At the same time, the physiological parameter P1 is compared with the standard physiological parameters in the distinguishing database 231 to find out whether the physiological parameter P1 is normal or abnormal.

In the event, the physiological parameter P1 is normal, the processor 21 will store the physiological parameter P1 within the memory device 23. In the event, the processor 21 judges that the physiological parameter P1 is abnormal after comparing with the standard physiological parameters in the distinguishing database 231, the abnormal physiological parameter P2 is transmitted to the micro processing unit 11 via the wireless reader 22 and the wireless transceiver module 13 so as to activate the alarm device 14.

At the same time, the processor 21 compares the abnormal physiological parameter P2 with those in the prescription database 232, where, the abnormal physiological parameter P2 is displayed in the display unit 24 so as to remind the ambient persons or the patient 10 to take life saving treatment. When the physiological parameter is abnormal, the processor 21 will select a respective prescribed treatment from the prescription database 232 in response to the abnormal physiological parameter P2 after comparing the latter with respect to the standard physiological parameters. The processor 21 will transmit the abnormal physiological parameter P2, the normal physiological parameter P1 and the respective prescribed treatment to the distal recipient 3 via the transmitting unit 25.

In the present invention, the transmitting unit 25 is connected to the distal recipient 3 via the Internet. Preferably, the distal recipient 3 can be a PC (Personal Computer) installed in a medical center or hospital. The real-time diagnose device 2 can also be a PC (Personal Computer).

From the above-mentioned explanation, one can note that the diagnose system of the present invention is applied in a condition for capturing the physiological signals of several persons 10, 10a, 10b for analyzing the same to judge whether the signals are normal, thereby effectively providing a respective prescribed treatment in time of critical and emergency time.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A diagnose system comprising:
a signal-capturing device including
a signal-receiving interface for acquiring a physiological signal of a person under observation,
a micro processing unit coupled electrically to said signal-receiving interface for processing said physiological signal and promptly converting said physiological signal into a physiological parameter, and
a wireless transceiver module coupled electrically to said micro processing unit for transmitting said physiological parameter;
a real-time diagnose device coupled electrically to said signal-capturing device and including
a wireless reader coupled electrically to said wireless transceiver module for receiving said physiological parameter,
a memory unit including a distinguishing database and a prescription database, said distinguishing database being stored with a plurality of standard physiological parameters as reference for judging whether said physiological parameter is normal or abnormal, said prescription database being stored with a plurality of prescribed treatments, one of which is corresponding to said physiological parameter in case the latter is abnormal, and
a processor coupled electrically to said wireless reader and said memory unit for promptly analyzing said distinguishing database, thereby distinguishing said physiological parameter received in said wireless reader;
wherein, in case said physiological parameter is abnormal, said processor will select a respective prescribed treatment from said prescription database in response to said abnormal physiological parameter after comparing said abnormal physiological parameter with respect to said standard physiological parameters.

2. The diagnose system according to claim 1, wherein said real-time diagnose device further includes a display unit coupled electrically to said processor for displaying said respective prescribed treatment and said physiological parameter received in said wireless reader.

3. The diagnose system according to claim 1, wherein said signal-capturing device further includes an alarm device coupled electrically to said micro processing unit.

4. The diagnose system according to claim 3, wherein said processor transmits said abnormal physiological parameter to said micro processing unit via said wireless reader and said wireless transceiver module so that said micro processing unit activates said alarm device.

5. The diagnose system according to claim 1, wherein said real-time diagnose device further includes a transmitting unit coupled electrically to said processor.

6. The diagnose system according to claim 5, wherein said processor transmits said physiological parameter, said abnormal physiological parameter and said respective prescribed treatment to a distal recipient via said transmitting unit.

7. The diagnose system according to claim 6, wherein said transmitting unit is connected to said distal recipient via the Internet

8. The diagnose system according to claim 6, wherein said distal recipient is a PC (Personal Computer) installed in a medical center.

9. The diagnose system according to claim 1, wherein said real-time diagnose device is a PC (Personal Computer).

10. The diagnose system according to claim 1, wherein said wireless transceiver module is a Radio Frequency Modem.

11. The diagnose system according to claim 1, wherein said wireless reader is a wireless frequency reader.

12. The diagnose system according to claim 1, wherein said physiological signal is an ECG (Electrocardiogram) signal.

13. The diagnose system according to claim 1, wherein said physiological signal is a blood pressure of the person under observation.

14. The diagnose system according to claim 1, wherein said physiological signal is an amount of blood glucose of the person under observation.
